(19) United States
(12) Patent Application Publication
(10) Pub. No.: US 2003/0046725 A1
(43) Pub. Date: Mar. 6, 2003

(54) Method of organelle transformation

(76) Inventor: Charles P. Downer JR., Lubbock, TX (US)

Correspondence Address: Aaron R. Clements, Hurley, Reyes & Guinn, 1805-13th Street, Lubbock, TX, 79401, US

(21) Appl. No.: 09/935,084

(22) Filed: Aug. 21, 2001

(51) Int. Cl. C12N 15/82; A01H 5/00; C12N 15/84

(52) U.S. Cl. 800/278; 435/468; 435/470; 800/293; 800/303

(57) ABSTRACT

A method for organelle transformation is disclosed in which a transgene-carrying organelle is physically transferred from a donor to a recipient, wherein the donor and the recipient may be of similar or dissimilar species. The method disclosed comprises isolation and concentration of the organelle to be transferred from the tissue of the donor followed by injection of the organelle into the cytoplasm of a target organism, whereupon the transgene of the injected organelle is expressed.

### CROSS REFERENCE TO RELATED APPLICATIONS

Not Applicable.

### STATEMENT REGARDING FEDERALLY SPONSORED RESEARCH

Not Applicable.

### REFERENCE TO “MICROFICHE APPENDIX”

Not Applicable.

### BACKGROUND OF THE INVENTION

This Invention relates to a method of organelle transformation involving the physical transfer of organelles, including mitochondria and plastids, between cells of similar or dissimilar species in order to quickly and efficiently transfer traits associated with cytoplasmic DNA to a target cell or organism. This method has the potential to reduce or eliminate the need for time-, labor- and cost-intensive backcrossing breeding methods which have traditionally been used to breed new traits into modified species, particularly agronomically important species.

Backcross breeding is a widely used method involving repeated subsequent generation crossing of the F₁ progeny of a hybrid crossing with its recurrent parent to attempt to recover and reinforce the desirable traits of the recurrent parent while retaining the desirable traits introduced by hybridization. These methods are particularly used in agricultural plant breeding to obtain new specimens with yields or other desired traits of the recurrent parent with new traits such as disease resistance. Backcross breeding is also used extensively in hybrid breeding programs specifically to transform cytoplasmic male sterility (CMS) between breeding lines. CMS allows breeders an additional measure of control in breeding programs by eliminating the possibility that a hybrid can self-pollinate, rather than be pollinated by the recurrent parent.

Despite the wide traditional use of this technique, backcross breeding suffers from a number of drawbacks. To effectively recover the genetic identity of the recurrenet parent, a successful backcross breeding program typically requires five to seven backcross generations, followed by several additional generations of selection. Each backcross generation requires hundreds of progeny plants in order to effectively screen and select for transgene expression and parental phenotype. Additionally, true to type parental genotypes are frequently not recovered in trangenic backcross breeding. The progenies of backcross breeding are therefore seldom equivalent agronomically to their recurrent parent. Part of the poorer agronomic performance of transformed varieties is due to the transformation process itself, but a substantial proportion of this problem is also inherent to the backcross breeding methods used to integrate desired traits into existing varieties. Problems associated with backcross breeding include the inheiritance of inferior traits of poor agronomic varieties used as trait donor parents; an inability to select for agronomic traits and parental types during the backcross process; and early accidental random selection of variant types expressing transgene traits. Furthermore, because of limited resources, backcrossing and trait screening is often carried out on a minimum scale, with only enough crosses made during each backcross generation to adequately screen for the transgene. The scale of any backcrossing project and limited resources available also prevent generation and selection of sufficient numbers of progenies great enough to sample and recover recurrent parent types. Overreaching all of these problems is the fact that backcross breeding can only be accomplished between similar species capable of sexual reproduction, for reasons that are apparent.

In addition to the limitations of backcross breeding, which is used to select and reinforce the traits of the desired parental genotype, current methods for accomplishing transformation itself are currently lacking. In plant transformation, current methods are centered around a method known in the art as agrobacterium mediated transformation. This method involves insertion of single copies of strands of DNA into the nuclear genomes of plant cells, using a bacterium as a vector to carry the DNA. Despite the wide use of this technique, its inaccuracy, instability, and uncertain levels of single transgene expression in plants resulting from its use are less than satisfactory and create additional obstacles to the usefulness of transgenic plants where agronomic performance, gene product expression, and breeding methods are important. The agronomic performance of superior breeding lines can be degraded by the unintentional transformation of unwanted DNA carried along with desired genetic material during transformation processes, or from DNA contributed by donor parents during backcross breeding. In either case, the transformed varieties frequently fail to perform at the same agronomic level as their conventional recurrent parent due to unintended interactions with and disruption of the nuclear genome caused by transformation methods. In short, current transformation and backcross methods are inherently unstable, unpredictable, and resource inefficient (particularly in light of the uncertain outcomes of these methods). A number of publications discuss these problems and techniques, including the following sources: Firoozabody E, DeBoer D, Merlo D, Halk F, Amerson L, Rashka K, & Murray E (1987), Transformations of cotton (Gossypium hirsutum l.) by Agrobacterium tumefaciens and regeneration of transgenic plants, 10 Plant Molecular Biology 105-116; Sanford J. C., The Biolistic Process: Trends in Biotechnology (1988), pp. 299-302; Christou, P., McCabe, D. E., and Swain, W. F., Stable transformation of soybean callus by DNA-coated gold particles. 87 Plant Physiology 671-674 (1988); Genetic Drift Newsletter, Mitochondrial Inheritance 10: Winter 1994, www.mostgene.org/gd/gdvol10b.html; M. Kirkpatrick & Lande, R., The Evolution of Maternal Inheritance, 43 Evolution 485-503; J. M. Poehlman, Breeding Field Crops, Third Ed. (1988), p. 563; J. Song & Hedgcoth C., Influences of nuclear background on transcription of a chimeric gene (orf256) and coxI in fertile and cytoplasmic male sterile wheats. 37 Genome 203-209 (1994); Z. Svab, Hajdukiewicz, P., & Maliga, P., Stable transformation of plastids in higher plants, 87 Proc. Natl. Acad. Sci. USA 8526-8530 (1990); P. Maliga, Carrer, H., Kanevski, I., Staub, J., & Svab, Z., Plastid engineering in land plants: a conservative genome is open to change, 342 Philos. Trans. R. Soc. London B Biol. Sci. 203-208 (1993); D. R. Pring, Chen, W., Tang, H. V., Howad, W., & Kempken, F., Interaction of mitochondrial RNA editing and nucleolytic processing in the restoration of male fertility in sorghum. 33 Curr Genet 429-436 (1998); R. G. S. Bidwell, Plant Physiology, Second Ed. (1979), p. 55; Eichholtz, et al., U.S. Pat. No. 6,225,114; Reichert, et al., U.S. Pat. No. 6,153,813.

### SUMMARY OF THE INVENTION

The present Invention involves a method for transforming a cell's cytoplasmic genome through introduction of a genetically modified organelle using a conceptually simple process. First, a genetically modified organelle (or an organelle carrying a gene for a desired naturally-occurring trait) is isolated in a buffer solution. Following isolation, the organelle is then inserted into a target cell. The method of insertion can vary from implantation of the organelle vector at a cellular, microscopic level (in the case of individual cells) to a macroscopic insertion technique involving spraying a target organism (plant or otherwise) with a pressurized buffer solution containing the organelle vector. In either case, once the organelle vector has been inserted, its genome is immediately expressed in the target. These and other features, aspects, and advantages of the present invention will become better understood with reference to the following description and appended claims.

### DESCRIPTION

A method according to the present invention for achieving organelle transformation comprises the steps of isolating and concentrating an organelle containing a transgene which expresses a desired trait in a buffer solution followed by spraying a target cell, tissue, or organism with the buffer solution under pressure in order to insert the organelle through the target's cell membrane and cell wall. After insertion of the organelle into the target cytoplasm, the transgene begins to express the desired trait immediately.

In the isolation and concentration step, one begins with a donor organism which has organelles carrying the desired transgene. Tissue from this organism is broken down in a buffer solution, resulting in a mixture of cellular parts, including whole organelles carrying the desired trangene. While they must be transferred to a target to survive, the autonomous organelles can survive in this buffer solution for some time. The buffer solution is then filtered through a selective medium which separates the cellular parts by their particle size. The fraction of the buffer solution containing particles of a size comparable to that of the desired transgene carrying organelle is separated out, and this filtrate is then used in the insertion step. This filtration process not only results in eliminating many undesired fragments of the original tissue, but it also results in substantial concentration of the desired transgene carrying organelle, simply because a large volume of the buffer solution is discarded with the undesirable fractions. Other methods of isolating the desired organelle are conceivable, including direct extraction of the desired organelle from the cytoplasm of cells of the donor organism. However, the bulk extraction process is much easier and cost-effective, at least in the case of plants.

In the insertion step, the filtrate from the isolation and concentration step is mixed with an abrasive such as fine silica gel. This mixture is then sprayed under pressure using a standard pneumatic spray gun onto healthy plants. The abrasive serves to erode the cell walls of the target plants, which enables the desired organelle to more easily penetrate through to the cytoplasm of the target plant's cells without damage. Upon insertion, the desired organelle begins to express its genome.

To test this method, the organelle and desired trait selected were mitochondria from cotton plants, Gossypium sp., exhibiting cytoplasmic male sterility, which is a known mitochondrial trait. Mitochondria were extracted from male sterile Gossypium harknesii and concentrated by the methods described above. They were then applied by the insertion method described above to healthy, male fertile plants of Gossypium hirsutum. As an additional test to demonstrate the effectiveness of this technique, untreated and buffer only controls were also sprayed onto plants to quantify enviromental sterility and sterility caused by physical damage to plants during the insertion process.

Beginning twenty-four days after treatment and continuing for six days during the peak bloom cycle of the test plants, the frequency of complete and partial sterility within the treated population averaged several times greater than in either control treatment. The data are summarized in the following table, which demonstrates statistically the efficacy of this method (numbers indicate the number of sterile blooms observed among samples of 250 plants for each control group):

TABLE 1
Frequency of Stable Blooms based on treatment type.

| Days After | Mitochondria | Buffer and | Untreated |
|---|---|---|---|
| Treatment | Buffer Solution | Abrasive only | Control |
| 17 | 3 | 4 | 4 |
| 18 | 5 | 5 | 3 |
| 19 | 6 | 4 | 5 |
| 20 | 6 | 5 | 3 |
| 21 | 5 | 4 | 5 |
| 22 | 7 | 5 | 6 |
| 23 | 9 | 6 | 3 |
| 24 | 17 | 8 | 4 |
| 25 | 42 | 7 | 6 |
| 26 | 33 | 8 | 7 |
| 27 | 29 | 9 | 5 |
| 28 | 21 | 7 | 6 |
| 29 | 10 | 5 | 4 |
| 30 | 8 | 4 | 5 |
| 31 | 7 | 5 | 5 |
| 32 | 6 | 4 | 4 |
| 33 | 8 | 6 | 5 |

During days 24-29, a significantly larger than average number of sterile blooms were observed, indicating that despite some plant damage and mortality as well as environmental factors and natural occurrence of the cytoplasmic male sterility trait, the method of the present invention is effective in transforming cells in the target plants.

Overall, the previously described method of the present invention shows substantial advantages over traditional methods of both nuclear- and organelle-based transformations. This version is cheap, cost-effective, and requires little specialized equipment outside that ordinarily found in a general laboratory. Furthermore, this version of the present invention produces rapid results, with the transgene beginning to express within the time span of a bloom cycle in the initially treated generation, without altering the agronomic properties of the initially treated generation. Because the original agronomic properties are preserved in the transformed specimen, the need for multiple backcrossing generations is eliminated, substantially accelerating the development of the new plant variety. Of course, the advantages of this method are dependent on the transgene, organelle, and donor species selected for involvement in the transformation process, and one or more of these advantages may be impacted with changes in these variables.

Although the present invention has been described with reference to certain preferred versions thereof, other versions are possible. Therefore, the spirit and scope of the appended claims should not be limited to the description of the preferred versions contained herein. Any element in a claim that does not explicitly state “means for” performing a specified function, or “step for” performing a specific function, is not to be interpreted as a “means” or “step” clause as specified in 35 U.S.C. §112(6).

The invention claimed is:

1. A method of organelle transformation comprising:
a. a method of isolation of an organelle carrying a gene for a desired trait;
b. a method of physically inserting said organelle into target cells.

2. The method of claim 1, wherein said method of isolation further comprises:
a. a method of breaking down cells containing said organelle in a buffer solution such that said buffer solution contains particles which are fragments of said cells;
b. filtering said buffer solution through a selective matrix that allows particles in said buffer solution to pass through said matrix at different rates; and
c. retaining the portion of the filtrate of said buffer solution containing particles of a size comparable to that of said organelle.

3. The method of claim 1, wherein said method of physically inserting said organelle further comprises:
a. isolation of said organelle in a buffer solution;
b. spraying said buffer solution containing said organelle under pressure onto target cells.

4. The method of claim 1, wherein said method of isolation further comprises:
a. a method of breaking down cells containing said organelle in a buffer solution such that said buffer solution contains particles which are fragments of said cells;
b. filtering said buffer solution through a selective matrix that allows particles in said buffer solution to pass through said matrix at different rates; and
c. retaining the portion of the filtrate of said buffer solution containing particles of a size comparable to that of said organelle;
and wherein said method of physically inserting said organelle into target cells further comprises spraying said portion of the filtrate under pressure onto target cells.

5. The method of claims 1, 2, 3, or 4 wherein said target cells are plant cells.

6. The method of claim 5 wherein said organelle is a mitochondrion.

7. The method of claim 6 wherein said mitochondrion carries genes which expresses the trait cytoplasmic male sterility.

8. An organelle transformed plant produced by the method of claims 1, 2, 3, or 4.

9. An organelle transformed plant produced by the method of claim 5.

10. An organelle transformed plant produced by the method of claim 6.

11. An organelle transformed plant produced by the method of claim 7.
